(19) United States
(12) Patent Application Publication
(10) Pub. No.: US 2017/0014209 A1
(43) Pub. Date: Jan. 19, 2017

(54) Periodontal Subperiosteal Tunnel Bone Graft Technique

(71) Applicant: Sung Joo Jeong, Paoli, PA (US)

(72) Inventor: Sung Joo Jeong, Paoli, PA (US)

(21) Appl. No.: 15/096,331

(22) Filed: Apr. 12, 2016

Related U.S. Application Data
(60) Provisional application No. 62/191,542, filed on Jul. 13, 2015.

(51) Int. Cl. A61C 8/02; A61B 17/04; A61B 17/16

(52) U.S. Cl. CPC A61C 8/0006; A61B 17/1673; A61B 17/04

(57) ABSTRACT

Tunnel bone grafting according to the proposed method can be used when an individual has a facial bone defect due to periodontal disease, cleft palate or trauma. The proposed method can be utilized for implant placement, it reduces wrinkles and supports facial muscle in order to make the individual look younger. The method can also be used to create a thicker alveolar ridge in order to stabilize full dentures or partial dentures. Its advantages over conventional guided bone regeneration techniques is that the proposed method is minimally invasive and lessens trauma to patients, prevents soft tissue opening, and reduces surgery time. Specifically, it minimizes the incision size and thus reduces trauma to the patient. Further, this reduces the risk of complications, including excessive bleeding, due to the minimization of the incision size. There is also considerable reduction in post surgery swelling using the proposed method as opposed to conventional techniques.

### CROSS-REFERENCE TO RELATED PATENT APPLICATIONS

This application claims priority under 35 U.S.C. Section 119(e) from U.S. Provisional Application Number 62/191,542, filed Jul. 13, 2015, entitled “Periodontal Subperiosteal Tunnel Bone Graft Technique,” the entirety of which is incorporated herein by reference.

### BACKGROUND OF THE INVENTION

Field of the Invention

The present invention relates generally to a method of performing periodontal surgeries to correct damage or defects to facial bones due to periodontal disease, cleft palate or trauma.

Related Art

Trauma can cause dimensional changes to the hard and soft tissues of the mouth and face and irregularities in the smooth contours of the cheek. Gum disease can cause bone loss in the alveolar ridge in the mouth and loss of teeth. Also, cleft palate causes a lack of alveolar bone and missing teeth in some cases if the cleft extends through the alveolus.

Untreated gingivitis can advance to periodontitis. With time, plaque can spread and grow below the gum line. Toxins produced by the bacteria in plaque irritate the gums. The toxins stimulate a chronic inflammatory response in which the body in essence turns on itself, and the tissues and bone that support the teeth are broken down and destroyed. Gums separate from the teeth, forming pockets (spaces between the teeth and gums) that become infected. As the disease progresses, the pockets deepen and more gum tissue and bone are destroyed. As bone is destroyed, eventually teeth can become loose and may have to be removed.

Many individuals that suffer tooth loss due to periodontitis, trauma, or cleft palate may desire implants. However, if bone mass is lacking, it must be replaced prior to the placement of implants. Without sufficient volume in bone mass, the placement of dental implants will not be successful. Therefore, renovation of alveolar bone mass is needed in order to allow for successful dental implantation.

The common approach to replacing lost bone mass is an invasive procedure with long recovery time, blood loss, swelling of the area of the face operated on, and severe pain caused to the patient. The gum is lifted away from the alveolar ridge to expose the inadequate or defective bone area. The area in need of bone mass is then filled with bone or bone substitute to build up the alveolar ridge. Sutures are placed, and after the lengthy healing process, a post normally of titanium is placed in the new bone mass to serve as the tooth's root. The implant fuses with bone, and replacement teeth are attached thereafter. The replacement is permanent.

The conventional method requires a large incision, which results in excessive bleeding because of an unnecessarily large tissue opening. Unnecessary swelling is also a common effect of the conventional method of replacing bone mass. Other effects in some cases include needless pain to the patient.

Due to the problems caused from conventional surgical methods described above, a minimally invasive alternative that accomplishes the same results is desirable. The method must be effective, minimally invasive, safe and performed without great difficulty.

### BRIEF SUMMARY OF THE INVENTION

A 6 mm to 10 mm, but at the most 15 mm, vertical incision is made beyond the mucogingival junction at the mesial side of the area that needs horizontal bone augmentation. Then, periosteal elevators are used to separate the periosteum from alveolar ridge on the buccal side. The alveolar bone area is scraped using a back action chisel or small head bone file. A collagen membrane is inserted inside of the tunnel. Then, a freeze dried bone allograft cortical or cortical cancellus powder mixed with saline solution or the patient's blood is placed between the collagen membrane and the alveolar bone surface in order to fill the void created. The incision is sutured and six months elapses prior to placing implant.

### BRIEF DESCRIPTION OF THE DRAWING

FIG. 1 shows a flow-chart illustrating the steps included in performing the Periodontal Subperiosteal Tunnel Bone Graft Technique.

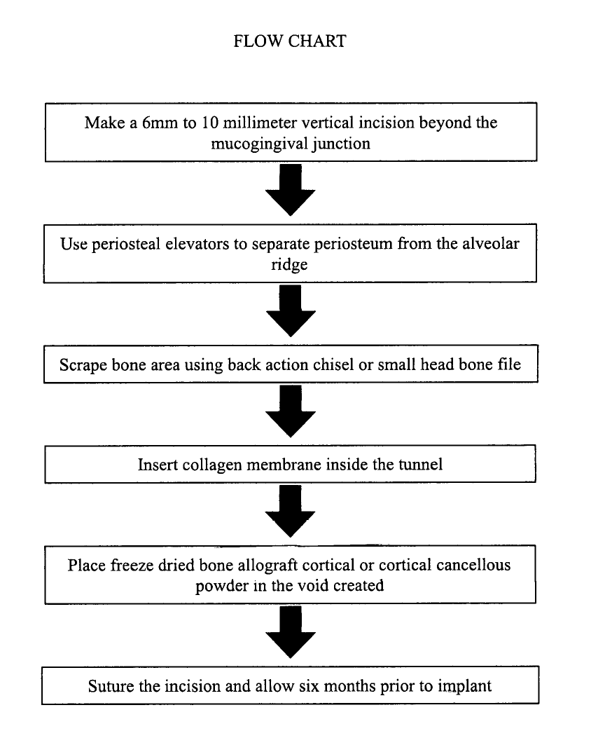

### DETAILED DESCRIPTION OF THE INVENTION

The present invention relates generally to methods of performing periodontal surgeries. It is a method of performing periodontal surgery in order to correct damage to the alveolar ridge that has decreased bone mass to such a degree that it is not medically advisable to place dental implants. The sources of damage could include trauma, facial bone defects due to periodontal disease, or cleft palate.

A 6 mm to 10 mm, but at the most 15 mm, vertical incision is made beyond the mucogingival junction at the mesial side of the area that needs horizontal bone augmentation. Then, periosteal elevators are used to separate the periosteum from alveolar ridge on the buccal side. The alveolar bone area is scraped using a back action chisel or small head bone file. A collagen membrane is inserted inside of the tunnel. Then, a freeze dried bone allograft cortical or cortical cancellus powder mixed with saline solution or the patient's blood is placed between the collagen membrane and the alveolar bone surface in order to fill the void created and to supplement the area with decreased bone mass. The incision is sutured. The patient is given approximately six months of time to heal and for the bone graft to convert into living bone. Afterwards, the implant can be placed using accepted conventional methods.

One variation on the within method includes, at the time of placing the sutures, also placing an external mattress suture over the recipient site to stabilize the graft area.

1. A method of performing periodontal surgeries in order to correct damage to the alveolar ridge that has decreased bone mass to such a degree that it is not medically advisable to place dental implants, which comprises the following steps: making a vertical incision of 6 mm to 10 mm, but at the most 15 mm, beyond the mucogingival junction at the mesial side of the area that needs horizontal bone augmentation, placing periosteal elevators to separate the periosteum from alveolar ridge on the buccal side, scraping the alveolar bone area using a back action chisel or small head bone file, inserting a collagen membrane inside of the newly created tunnel, placing a freeze dried bone allograft cortical or cortical cancellus powder mixed with saline solution or the patient's blood between the collagen membrane and the alveolar bone surface in an amount sufficient to fill the void created, suturing the incision, waiting approximately six months prior to placing the dental implant

2. A method of performing periodontal surgeries in order to correct damage to the alveolar ridge that has decreased bone mass to such a degree that it is not medically advisable to place dental implants according to claim 1, further comprising at the time of placing sutures, also placing an external mattress suture over the recipient site to stabilize the graft area.
